(19) United States
(12) Patent Application Publication
(10) Pub. No.: US 2024/0307579 A1
(43) Pub. Date: Sep. 19, 2024

(54) Laser virus filter screen

(71) Applicant: Xi LI, Shanghai (CN)

(72) Inventor: Xi LI, Shanghai (CN)

(21) Appl. No.: 17/442,126

(22) PCT Filed: Jul. 14, 2020
(86) PCT No.: PCT/CN2020/101768

§ 371 Date: Jun. 6, 2024

(51) Int. Cl. A61L 9/20; G01V 8/22

(52) U.S. Cl. CPC A61L 9/20; G01V 8/22; A61L 2209/111; A61L 2209/12

(57) ABSTRACT

A re-chargeable lithium battery drives a laser generator, and a laser is sent to a unidirectional beam expander by means of optical fiber. The widened and flattened laser beam enters a laser virus-filtering screen by means of a unidirectional fluoroscopy film. After laser reflections scan and cover the frame of the virus-filtering screen, the laser is extracted to a photoelectric sensor by means of a semi-transmissive film. If the energy of the extracted laser is less than a fixed value, suggesting that a foreign body has entered the laser virus-filtering screen and is blocking the laser, for safety reasons, the photoelectric sensor triggers a laser controller to immediately stop emitting the laser.

### TECHNICAL FIELD

Anti-epidemic.

### BACKGROUND TECHNIQUE

The laser has high intensity, tens of billions of times brighter than the sun, and has good directionality. The virus can be instantly killed by the ultraviolet laser's lytic effect on the virus or the infrared laser's heating effect.

### Technical Problem

Wearing a mask is sultry and cannot completely filter the virus, breathe hard, will inhale the exhaled air, feel uncomfortable, hinder conversation, and hinder recognition of human faces.

### Technical Solutions

The laser beam covers a flat or curved surface through multiple reflections, and the air passing through the surface will filter the viruses in it.

### Beneficial Effect

The laser virus filter screen can be used as a protective helmet, a mask, an air inlet and outlet of an air conditioner, and a large isolation screen in a room. The advantages of the laser virus filter screen are: it does not hinder the circulation of air, does not hinder the transmission of sound, does not block the light, and filters the virus in and out of the two-way direction. Completely complete invisible virus filtering.

The large-scale laser virus filter screen can be installed in the workplace with a laser beam expander to make the laser beam thicker to prevent mutual infection.

### DESCRIPTION OF THE DRAWINGS

The following is a description of the drawings:

1. Laser virus filtering square screen solution .dwg

(Attached drawing 1)

The white laser emitting head is connected to the laser generator by an optical fiber (incoherent laser) and installed at a certain angle. The blue line is the reflective surface, the white line is the beam reflected by the horizontal reflective surface just after the laser is launched, the beam reflected by the left vertical reflective surface is represented by a red line, and the beam reflected by the right vertical reflective surface is represented by a yellow line. The light beam reflected by the left vertical reflecting surface for the second time is represented by a green line, and the light beam reflected by the right vertical reflecting surface for the second time is represented by a cyan line, so that the laser beam densely covers the entire blue box and is always reflected in the frame . A photoelectric sensor is installed where the laser beam is densely covering the entire blue box. Part of the laser light can enter the photoelectric sensor through the semi-reflective surface. If foreign matter enters the box, the laser beam will stop emitting laser immediately when it encounters obstacles.

When the air passes through the blue box, the laser beam kills viruses in the air and becomes virus-free air.

2. The laser virus filter screen can be made into an arc shape. The curved surface is a part of the cylinder, the upper and lower reflecting surfaces are part of the conical surface coaxial with the cylindrical surface, and the left and right reflecting surfaces are part of the plane passing through the axis of the cylinder. Like the plane laser virus filter, it is woven a dense shielding layer by multiple reflections of the laser. In the schematic diagram of laser virus filtering arc screen scheme .dwg:

(Attached drawing 2)

The yellow is the reflective frame, the red is the laser emitting head, the red line represents the path of the laser up and down reflection, and the blue line represents the path of the up and down reflection after the laser is reflected by the laser side reflecting surface . Repeated reflections on both sides. See the 3.dwg diagram of the conical surface reflection principle of the laser virus filter arc screen:

(Attached drawing 3)

The red is the laser beam, and the cyan is the normal of the laser reflection. The green line that passes through the cylinder axis and the reflection point and is perpendicular to the normal is the straight line generated by the conical surface. The upper and lower reflection surfaces are part of it. The cylindrical surface is mirror-symmetrical with respect to the magenta plane, and the left and right reflection surfaces are part of the plane. The reflection path of the laser beam by the plane and the path of removing the reflection surface are also in a mirror image relationship. So the laser is always reflected back and forth in this frame.

3. The principle of laser reflection scanning. Dwg description:

(Attached drawing 4)

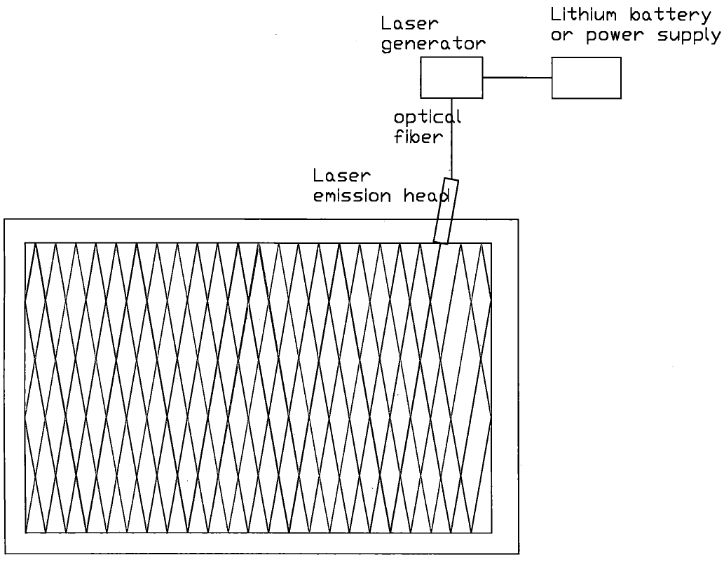

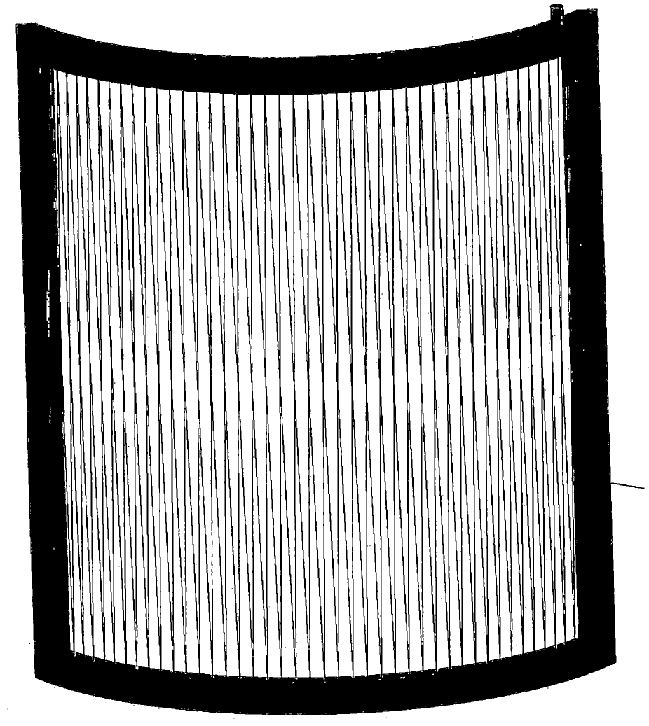

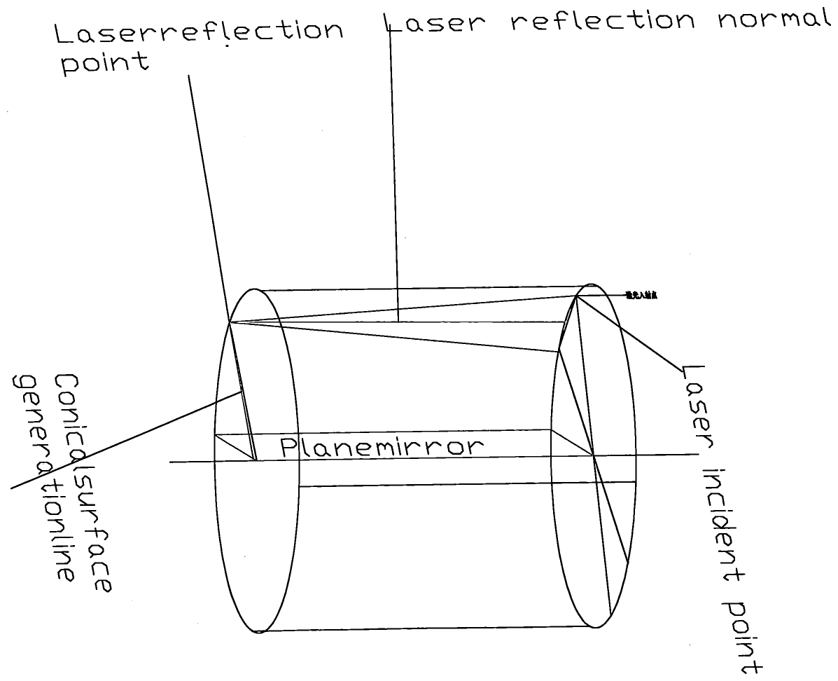

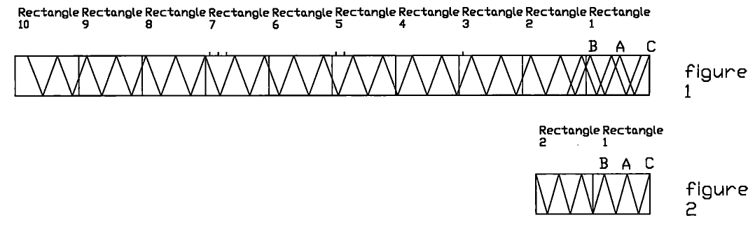

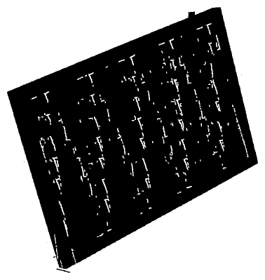

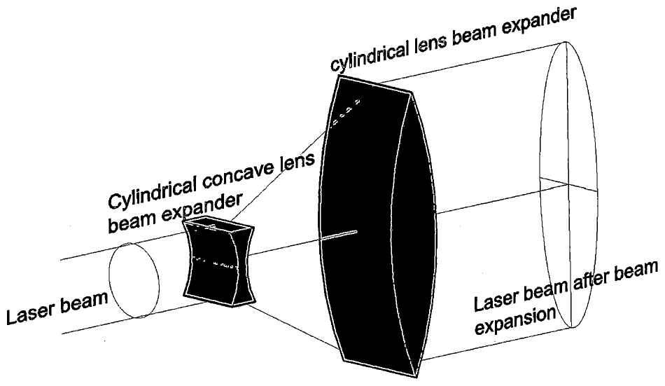

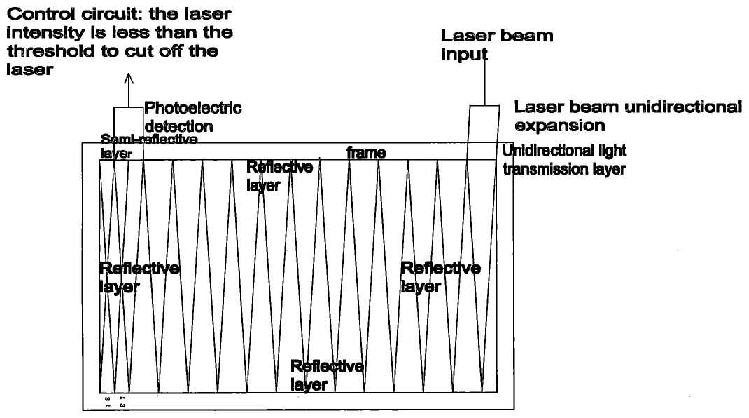

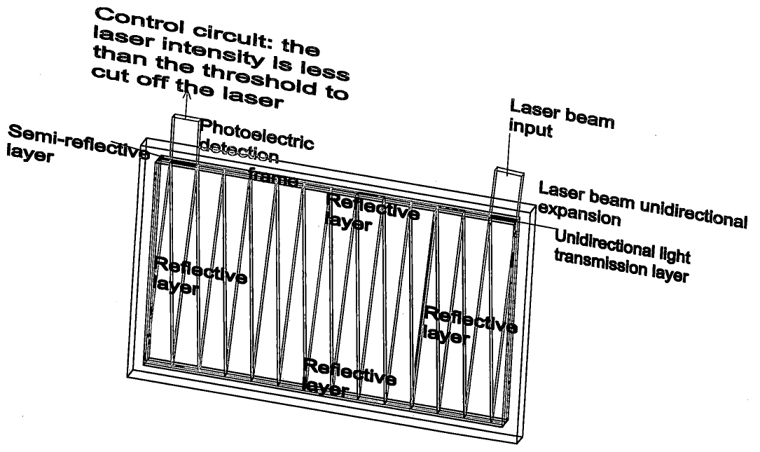

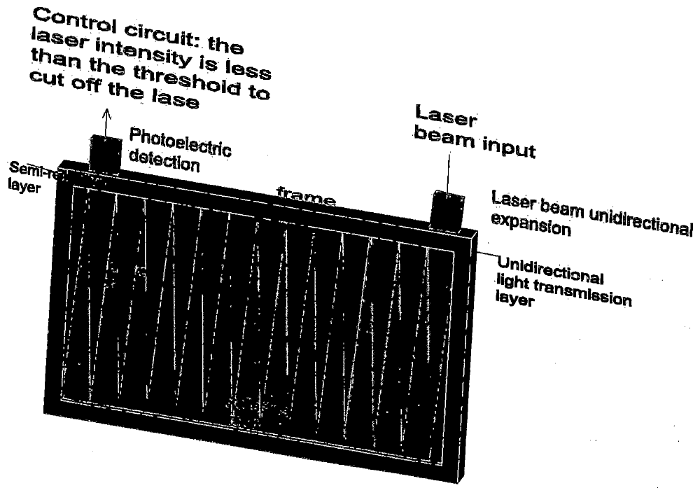

Because the left and right are flat mirrors, the image of the rectangular frame in the flat mirror can be regarded as a rectangular extension, because there are flat mirrors on the left and right, this extension can be regarded as infinite. See FIG. 1 of the laser reflection scanning principle. As long as the integral multiple of the distance from the reflective point A to the incident point C plus the radius or half of the width of the laser beam is exactly equal to the width of the rectangle, the laser beam will sweep and cover the entire rectangular frame. The blue and red slanted line in FIG. 1 represents the boundary of the laser beam. The result of the reflection scan is that rectangle 2 is folded to the right onto rectangle 1, rectangle 3 is folded to the left onto rectangle 2, rectangle 4 is folded to the right onto rectangle 3, and rectangle 5 is folded to the left onto the rectangle 4, the rectangle 6 is folded to the right onto the rectangle 5, and the rectangle 7 is folded to the left onto the rectangle 6. After the 7 rectangles overlap, the laser beam covers the entire rectangle. The same arc-shaped laser virus filter can be expanded into a plane, and the scanning process is the same. FIG. 2 is the case where the distance from the reflective point A to the incident point C is equal to the diameter or width of the laser. As long as the rectangle 2 is folded to the right onto the rectangle 1, the entire rectangle is covered.

4. 3D simulation of laser virus filter screen. dwg

(Attached drawing 5)

Simulated laser reflection scanning, red represents the laser emitting head, magenta represents the laser beam, its reflection is drawn according to the physical reflection theorem, after the first side plane reflection, the laser beam is shown in red, after the second side plane reflection, The laser beam is shown in yellow. After the third side plane reflection, the laser beam is shown in green. After the fourth side plane reflection, the laser beam is shown in cyan. After the fifth side plane reflection, the laser beam is shown in gray (250). Said and so on, and finally covered the entire rectangular box.

5. The laser beam can be changed from a round shape to a flat shape by beam expansion technology, as long as the spherical concave lens and spherical convex lens in the beam expander are changed to cylindrical concave lens and cylindrical convex lens, it can only be in the Y direction Expanding the beam does not change in the X direction, so that the laser beam becomes an ellipse approximately flat rectangle. The rectangular frame can be covered with a small number of reflections.

The principle of laser unidirectional beam expansion. Dwg:

(Attached drawing 6)

The cross section of the laser beam is circular. The beam expander uses a cylindrical concave lens instead of a spherical concave lens, and a cylindrical convex lens replaces a spherical convex lens. The beam expands only in one y direction, and remains unchanged in the x direction. After beam expansion, the laser beam is elliptical (or oblate). Greatly increase the width of the laser beam. Like FIG. 2 in the principle of laser reflection scanning. dwg, it greatly reduces the number of reflection to cover the filter box, which can reduce the reflectivity and manufacturing accuracy requirements of the filter box.

6. Laser beam expansion filtering .dwg:

(Attached drawing 7)

The function of the yellow parallelogram in the upper right corner of the figure is a laser beam expander, which makes the laser a wide and flat shape, which is represented by two parallel yellow lines and emits to the lower left. The laser beam reflected by the reflecting surface on the left is represented by two red lines. After multiple reflections up and down, it reflects from the left back to the right, covering the entire box. Laser beam expanding filter 3D.dwg

(Attached drawing 8)

The laser beam in is represented by yellow and red rectangles (because the elliptical cylinder is not easy to draw), and the simulated reflections are all drawn according to the physical reflection theorem.

(Attached drawing 9)

[symbol]

The best mode of the present invention

The laser generator is driven by a rechargeable battery, and the laser is sent to the laser unidirectional beam expander through an optical fiber. The widened and flattened laser beam enters the laser virus filter screen through the single transparent membrane, and covers the virus filter screen frame through reflection scanning Then, it is led to the photoelectric sensor through the semi-transparent membrane. If the laser energy is less than a fixed value, it means that the foreign matter enters the laser virus screen to block the laser. For safety, the photoelectric sensor triggers the laser controller to immediately stop emitting the laser.

### Embodiments of the Present Invention

The laser beam expands, enters the laser virus filter screen through a single transparent membrane, and covers the virus filter screen frame through reflection scanning.

### Industrial Applicability

The frame of the laser virus screen can be formed by hot pressing of transparent plastic, and the inside of the frame is coated with a reflective film and protected by a transparent plastic layer.

[symbol]

### Sequence Listing Free Content

The above drawings are all drawn by AUTOCAD, 3D, can be zoomed in, can be rotated, can be seen very clearly, and can be provided in the future.

[symbol]

1. Laser covers a plane or curved surface through multiple reflections, and the virus suspended by air passing through this plane or curved surface is killed and filtered.

2. In claim 1, the laser beam is unidirectionally expanded using cylindrical concave lenses, cylindrical convex lenses, or other methods, the cross-section changes from circular to elliptical or approximately rectangular, and a plane or curved surface is covered by multiple reflections, when air passes through this plane or curved surface, the virus suspended in it is killed and filtered.

3. Install photoelectric sensors at appropriate positions on the frame of the plane or curved surface in claim 1, If foreign objects enter the frame to block the laser, immediately stop laser emission to ensure safety.
